(19) United States
(12) Patent Application Publication
(10) Pub. No.: US 2009/0281638 A1
(43) Pub. Date: Nov. 12, 2009

(54) PROSTHETIC FOOT

(76) Inventor: Samuel Tourneux, Saint Julien les Rosiers (FR)

Correspondence Address: WESTERMAN, HATTORI, DANIELS & ADRIAN, LLP, 1250 CONNECTICUT AVENUE, NW, SUITE 700, WASHINGTON, DC, 20036, US

(21) Appl. No.: 12/306,795

(22) PCT Filed: Jun. 21, 2007
(86) PCT No.: PCT/FR07/51491

§ 371 Date: Dec. 29, 2008

(30) Foreign Application Priority Data
Jun. 30, 2006 (FR) 06 52727

(51) Int. Cl. A61F 2/66

(52) U.S. Cl. 623/55

(57) ABSTRACT

Prosthetic foot, of the type with energy recovery, comprising at least one spring blade (2) made of composite material fixed integrally to the ankle or tibia, or to a support (1) fixed to the ankle or tibia, and designed to permit dynamic set-down on the ground (S). It further comprises a rigid arm (3) connected to the blade (2) via a first connecting means (22), which permits a pivoting articulation of the arm (3) on the blade (2) on an axis transverse to the latter; and in that the rigid arm (3) has a part (31) designed to permit a substantially vertical set-down on the ground, preferably to the rear of the ankle or tibia; and in that, moreover, the rigid arm (3) and the blade (2) are connected via a second connecting means (32) arranged in an area distant from the first connecting means (22).

### BACKGROUND OF THE INVENTION

(1) Field of the Invention

This invention relates to the field of the prosthetic organs, and more particularly to that of the prosthetic feet.

(2) Description of the Prior Art

There are presently known prosthetic feet aimed at substituting natural feet. These prosthetic feet are essentially of two types, the rigid prosthetic feet, eventually provided with an articulation acting as an ankle, and the dynamic prosthetic feet.

The rigid prosthetic feet are obviously little efficient and uncomfortable in use, because they by far do not have the flexibility of the natural foot. In order to compensate for the latter, they are often associated with stops made of elastic material, but these stops essentially act as a damper, and do not allow an energy recovery, so that walking practice is little dynamic, and very wearisome.

The dynamic prosthetic feet consist of deformable single-bloc prosthetic feet. They are made of an elastically deformable material, such as a composite material of the carbon-fibre or fibreglass type. They are generally in the form of at least one spring blade rigidly secured to the tibia or the ankle, which extends forwards and/or rearwards. These dynamic prosthetic feet permit a nearly full recovery of the energy used to deform them.

Though these dynamic prosthetic feet have advantages with respect to the rigid prosthetic feet, they still have drawbacks, namely because they impart an unfolding of the step rather far different from the natural unfolding, namely at the striking of the heel. Indeed, according to the design of these prosthetic feet, either the heel is rigid and, namely during downhill walking, the bearer rests on the heel, the portion entering into contact with the ground with some delay due to the time required for carrying out the tipping over, which causes a lack of resting and an instability that can be prejudicial; either the heel is flexible, which flexibility is achieved through a jack, or and additional spring blade, which permits to increase the stability, but, on the other hand, causes some swaying, due to the vertical crushing of the heel.

Thus, from U.S. Pat. No. 6,767,370 is known a prosthetic foot that provides a solution for these various drawbacks. This prosthetic foot includes, on the one hand, a C-shaped heel having some elasticity and, on the other hand, a portion for resting on the ground, comprised of two spring blades mounted parallel to each other and made integral by at least one of their ends, and connected in the median area by a spacer movable longitudinally or having a variable compression resistance, so as to act on the dynamic characteristics of said resting portion, the displacement of said movable element or the change of its compression resistance being controlled by the crushing of said heel the effects of which are transmitted to said element. The element can be a spacer capable of being displaced by a rod, or an element inflatable under the action of a pump arranged in the heel.

Such a prosthetic foot has advantages with respect to those known from other documents, in that it permits a change of the rigidity of the portion resting on the ground depending on the level of crushing of the heel. On the other hand, it does not permit, like a natural foot, to act on the set-down on the ground immediately after the striking of the heel.

On the other hand, this prosthetic foot implements many moving, sliding and friction elements, which, besides the high manufacturing cost, requires, on the one hand, a particular development and, on the other hand, permanent maintenance.

From FR 2 839 443 is also known a self-propelling artificial foot with embedded energy. This shoe comprises two subsets that intersect and are hinged to each other through a transversal axis, while actuators are intercalated between each of their end portions, which actuators consist of inflatable bladders or the like. Alternately inflating and deflating the bladders causes a succession of scissor motions of the subsets likely to simulate the unfolding of the foot.

This artificial foot has however the drawback of being in addition of a complex implementation, of depending on actuators using an external energy source.

Presently, there exists no prosthetic foot permitting a striking of the heel that, while having a certain flexibility, generates the immediately set-down of the front portion of the foot.

### SUMMARY OF THE INVENTION

The object of this invention is to provide a prosthetic foot permitting to cope with the various above-mentioned drawbacks.

The prosthetic foot according to the invention is of the type with energy recovery, comprising at least one spring blade made of composite material fixed integrally to said ankle or to said tibia, and designed to permit a dynamic set-down on the ground, and which essentially further comprises a rigid arm connected to the blade through a first connecting means, which permits a pivoting articulation of said arm on said blade on an axis transverse to the latter; and wherein the rigid arm has a portion designed to permit a substantially vertical set-down on the ground, preferably to the rear of said ankle or of said tibia; and wherein, moreover, said rigid arm and said blade are connected by a second connecting means arranged in an area distant from said first connecting means.

According to a particular embodiment of the prosthetic foot according to the invention, the first connecting means directly connects the arm to the blade.

According to another particular embodiment of the prosthetic foot according to the invention, the first connecting means connects the arm to the support of the blade.

According to an additional feature of the prosthetic foot according to the invention, the first connecting means is in the form of a pivoting articulation means on a shaft.

According to another additional feature of the prosthetic foot according to the invention, the first connecting means is an elastic means.

According to another additional feature of the prosthetic foot according to the invention, the second connecting means is in the form of means capable of resting on the blade.

According to another additional feature of the prosthetic foot according to the invention, the second connecting means is in the form of elastic connecting means.

The elasticity of the connecting means, whether at the level of the first connecting means and/or of the second connecting means, has in addition the advantage of permitting an eversion/inversion motion.

According to another additional feature of the prosthetic foot according to the invention, it includes means capable of permitting to vary the distance separating the two connecting means.

By varying the lever arm on the blade, the forces exerted on the latter by said arm are changed, and the flexibility of the heel and the period during which the foot is into contact with the ground is adjusted at the same time.

According to another additional feature of the prosthetic foot according to the invention, it includes means capable of permitting to adjust the distance separating the blade support from the portion of the arm designed to rest on the ground.

Irrespective of the embodiment, the prosthetic foot according to the invention has many advantages with respect to the existing ones, namely by permitting a quicker and longer flat set-down of the foot, which generates a better stability of the patient, especially when passing on sloped surfaces, and a continuous transition on one and the same blade operating in both directions during the step.

The advantages and features of the prosthetic foot according to the invention will become clear from the following description referring to the attached drawing, which represents several non-restrictive embodiments of same.

### BRIEF DESCRIPTION OF THE DRAWINGS

In the attached drawing:

FIG. 1 represents a schematic side view of a first embodiment of the prosthetic foot according to the invention.

FIG. 2 represents a schematic view from above of the same prosthetic foot.

FIGS. 3a, 3b, 3c and 3d represent schematic side views of the same prosthetic foot during successive phases of walking.

FIGS. 4 and 5 represent schematic perspective views of variants of the prosthetic foot according to the invention.

FIG. 6 represents a schematic side view of another variant of the same prosthetic foot according to the invention.

FIG. 7 represents a schematic side view of another embodiment of the same prosthetic foot according to the invention.

### DETAILED DESCRIPTION OF THE PREFERRED EMBODIMENTS

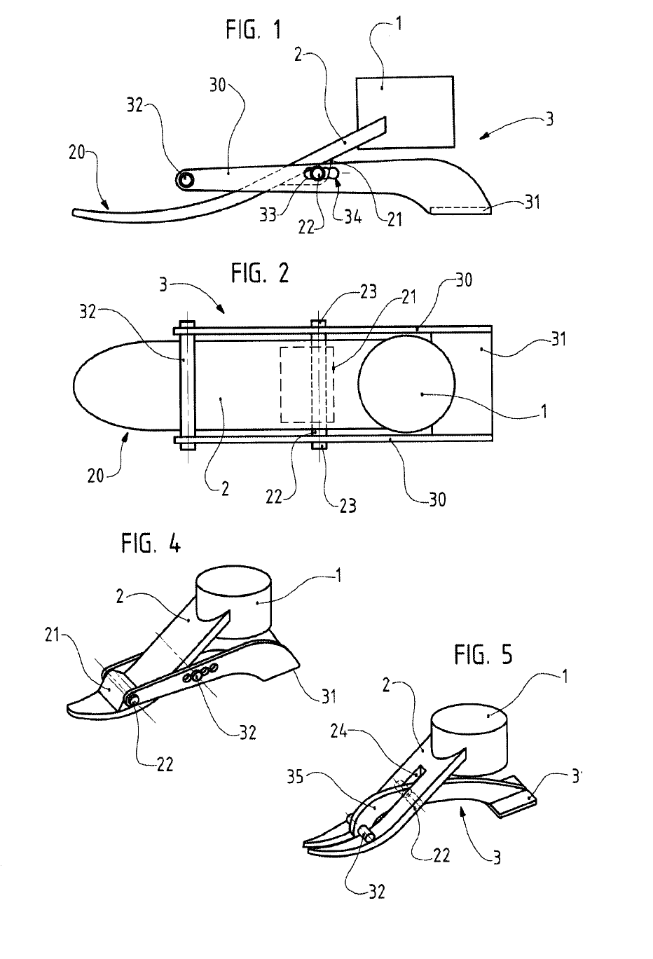

When referring to FIGS. 1 and 2, one can see a prosthetic foot according to the invention, which comprises a base 1 designed to be secured to the end of a leg, and to which is integrally fixed a spring blade 2 made of composite material, for example fibreglass. The spring blade 2 extends towards the front of the foot and according to a certain angle, so that its distal portion 20 can permit a dynamic set-down on the ground.

It should be noted that various configurations are possible at the level of the mounting of the prosthetic foot, for example, it can be contemplated to intercalate between same and the encasing of the tibia either rigid or flexible connecting means.

The spring blade 2 has in the lower portion of its median area a retaining plate 21 through which passes a transversal shaft 22, which consists of a first means for connecting to a rigid arm 3, which connection is brought about pivotally.

The arm 3 comprises two side flanges 30 each mounted on one end 23 of the shaft 22, and connected to the rear portion of the foot by a cross rod 31 and to the front portion by a transversal shaft 32 passing above the spring blade 2 and which constitutes a second connecting means.

The cross rod 31 is designed capable of constituting a support, and it forms in particular the heel of the prosthetic foot.

The shaft 22 passes through each of the flanges 30, through one of the holes 33 of a series 34 of holes distributed longitudinally, so that it is possible to choose to position the shaft 22 more or less separated from the shaft 32.

The arm 3 is pivotally movable with respect to the spring 2 blade 15, this pivoting is free, however limited in the direction of the extension of the foot by the transversal shaft 32 which abuts against the spring blade 2.

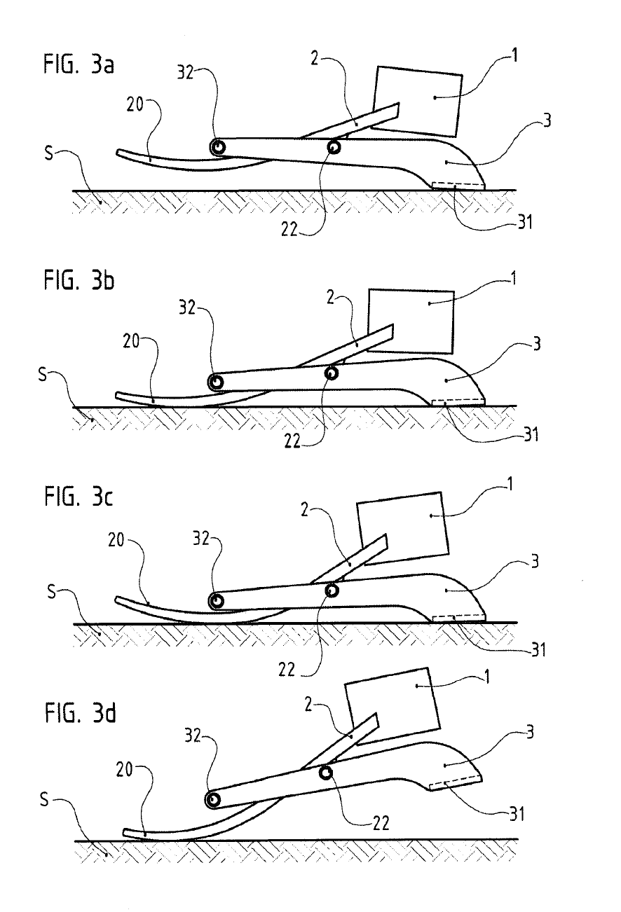

When referring now to FIGS. 3a, 3b, 3c and 3d, one can see the behaviour of the same prosthetic foot during walking practice.

In FIG. 3a, the prosthetic foot strikes against the ground S with the heel, i.e. with the cross rod 31. The force exerted on the base 1 causes the spring blade 2 to rise until it abuts against the transversal shaft 32, so that the base 1 is hanging from the end of the spring blade 2, therefore the set-down of the heel is dampened, while this causes the spring blade 2 to be applied against the ground S.

In FIG. 3b, the prosthetic foot stably rests on the ground S, both through the spring blade 22 and through the cross rod 31, the spring blade 2 being in addition braces against the shaft 32, so that the base 1 is hanging.

In FIG. 3c, the patient's weight passes towards the front, but the blade 2 and the heel 31 remain on the ground, thus providing a better stability.

In FIG. 3d, the foot rests on the front portion of the foot, thus only on the distal portion 20 of the spring blade 2, the arm 3 thus performs no action.

In this respect, it should be noted that restoring means can be provided for, which limit the angular opening between the spring blade 2 and the arm 3, more particularly the distance between the base 1 and the cross rod 31. The prosthetic foot is however designed to be contained in an esthetical casing, which can have this capacity of limiting the angular opening.

The prosthetic foot according to the invention thus has an unfolding very close to that of the natural foot. At the striking of the heel, there occurs a slight crushing of same, which crushing remains dynamic and generates as a reaction a quick entering into contact of the spring blade 2 with the ground, which effect is not possible with the existing prosthetic feet, such as the one from U.S. Pat. No. 6,767,370.

When referring now to FIGS. 4, 5 and 6, one can see variants of the prosthetic foot according to the invention.

In FIG. 4 is shown a prosthetic foot nearly identical to the one shown in FIGS. 1 to 3 and which differs from same only by the inversion of the connecting means, namely the shaft of pivoting 22 and the supporting transversal shaft 32, so that the articulation of the arm 3 is performed at the end of same, opposite to the cross rod 31. This prosthetic foot permits the same unfolding as the one shown in FIGS. 1 to 3.

In FIG. 5 is shown a prosthetic foot, which is also almost identical to the one shown in FIGS. 1 to 3, in which the shafts of pivoting 22 and support 32 are not inversed, but which differs in that the rigid arm 3 does not include two side flanges, but only one central body 35 that passes through a longitudinal slot 24 the spring blade 2 includes.

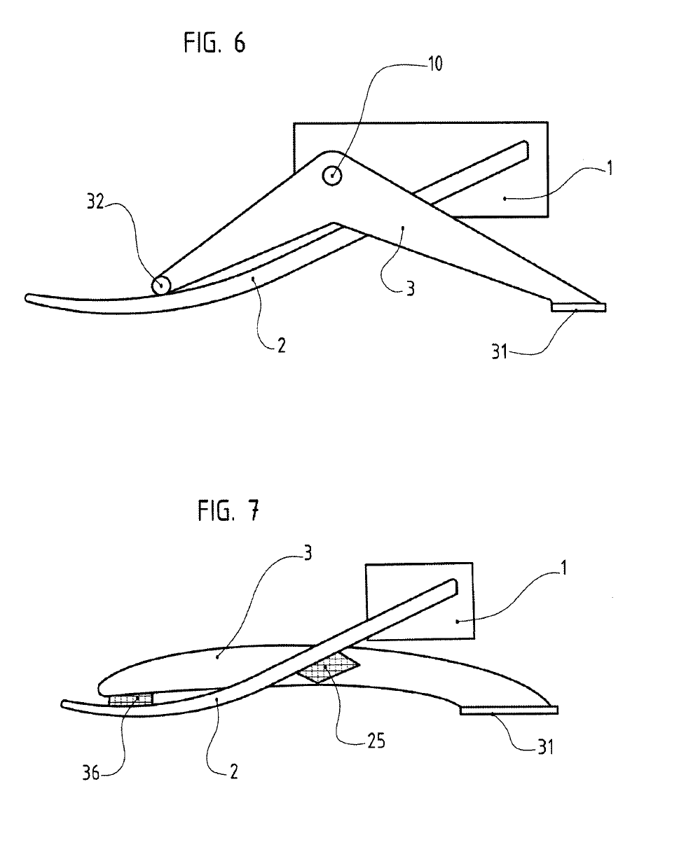

In FIG. 6 is shown another variant of the prosthetic foot according to the invention, wherein the rigid arm 3 is mounted pivotally, not directly on the blade 2, but on the base 1, through a shaft 10.

It should be noted that irrespective of the embodiment, there can be provided, besides the adjustment of the distance separating the shafts of pivoting 22, 10 and support 32, means permitting to adjust the distance separating the base 1 from the cross rod 31 or the like, i.e. to adjust the height of the prosthetic heel. This adjustment can be performed in various ways, such as, not restrictively, either a retaining plate 21 movable in longitudinal displacement alongside the blade 2 or a retaining plate 21 having a series of various holes for the passing through of the shaft 22, and extending in a direction not parallel to the general plane of the blade 2, or a series of holes in the base 1 for the passing through of the shaft 10, or a series of holes 33 extending in a direction not parallel to the main axis of the arm 3.

When referring now to FIG. 7, one can see another embodiment of the prosthetic foot according to the invention, wherein the arm 3 is connected to the blade 2 through, on the one hand, a first connecting means 25 consisting of an elastic connection that authorises a pivotal motion of the arm 3 about the blade 2; and, on the other hand, a second connecting means 36 arranged between the blade 2 and the end of the arm 3 opposite the resting portion 31, this second connecting means also being an elastic connection.

The connecting means 25 and 36 can consist of elements made of plastic, rubber or other materials having similar characteristics, made integral, for example through gluing, between the blade 2 and the rigid arm 3, or part of the latter.

These connecting means 25 and 36 can also be fixed to the blade 2 and/or to the rigid arm 3 through movable and removable means for making integral, in order to be able to change the distance separating them.

Such an embodiment permits to more closely connect the blade 2 and the rigid arm 3, and permits some flexibility in the traverse direction, and permits an eversion/inversion motion.

1. Prosthetic foot, of the type with energy recovery, comprising at least one spring blade made of composite material fixed integrally to the ankle or to the tibia, or to a support fixed to said ankle or said tibia, and designed to permit a dynamic set-down on the ground, which essentially further comprises a rigid arm connected to said blade through a first connecting means, which permits a pivoting articulation of said arm on said blade on an axis transverse to the latter; and wherein said rigid arm includes a portion designed to permit another substantially vertical set-down on the ground, preferably to the rear of said ankle or of said tibia; and wherein, moreover, said rigid arm and said blade are connected by a second connecting means arranged in an area distant from said first connecting means.

2. Prosthetic foot according to claim 1, wherein the first connecting means connects the arm directly to the blade.

3. Prosthetic foot according to claim 1, wherein the first connecting means connects the arm to the support of the blade.

4. Prosthetic foot according to claim 1, wherein the first connecting means is in the form of a pivoting articulation means on a shaft.

5. Prosthetic foot according to claim 1, wherein the first connecting means is an elastic connecting means.

6. Prosthetic foot according to claim 1, wherein the second connecting means is in the form of a means capable of resting on the blade.

7. Prosthetic foot according to claim 1, wherein the second connecting means is in the form of an elastic connecting means.

8. Prosthetic foot according to claim 1, which includes means capable of permitting to vary the distance separating the two connecting means.

9. Prosthetic foot according to claim 1, which includes means capable of permitting to adjust the distance separating the support of the blade with respect to the portion of the arm designed to rest on the ground.
